(19) United States
(12) Patent Application Publication
(10) Pub. No.: US 2017/0136080 A1
(43) Pub. Date: May 18, 2017

(54) NOVEL METHOD FOR THE TOPICAL APPLICATION OF VETERINARY AGENTS

(71) Applicant: Ceva Santé Animale, Libourne (FR)

(72) Inventors: Florence Guimberteau, Montussan (FR); Hamadi Karembe, Libourne (FR); Vassilios Kaltsatos, Libourne (FR); Gautier Barthelemy, Bordeaux (FR)

(73) Assignee: Ceva Santé Animale, Libourne (FR)

(21) Appl. No.: 15/322,805

(22) PCT Filed: Jun. 30, 2015
(86) PCT No.: PCT/EP2015/064832

§ 371 Date: Dec. 29, 2016

(30) Foreign Application Priority Data
Jun. 30, 2014 (EP) 14306049.9

(51) Int. Cl. A61K 36/53; A61Q 19/00; A61K 8/37; A61K 8/97; A61K 47/14; A61K 9/00

(52) U.S. Cl. CPC A61K 36/53; A61K 47/14; A61K 9/0017; A61K 8/37; A61K 8/97; A61Q 19/00; A61K 9/0087; A61K 2800/70

(57) ABSTRACT

The invention relates to compositions and to the methods of administration thereof. More specifically, it relates to an optimised method for the administration of active agents to non-human mammals by means of topical application of pre-determined doses.

The present invention relates to methods, apparatuses and compositions for the topical application of veterinary agents. It relates more specifically to methods, apparatuses and compositions which allow the topical and sequential application of veterinary compounds for ensuring better treatment adherence. The invention is applicable to any veterinary agent, in particular with a therapeutic or cosmetic or dermatological effect, and to any animal, preferably any non-human mammal.

### BACKGROUND OF THE INVENTION

Veterinary treatments are provided in various forms and mainly in injectable form or in the form of shampoos, of sprays, or of a deposit on the skin.

Shampoo treatments require application over the entire surface of the animal's skin and a step of rinsing with water, which is often unpleasant for said animal. In addition, treatments using shampoos cannot be applied to all therapeutic agents and provide only a short-term and transient treatment.

The application of spray treatment can prove to be quite complicated, since many animals find the noise or the odor of the spray objectionable. Furthermore, it is necessary to count the number of presses in order to adapt the dosage regimen of the weight of the animal treated and to take care to apply the product uniformly over the whole of the body. It is also necessary to apply the treatment 48 hours away from any bath. Finally, once the animal has been treated by spray, the coat of the latter often takes on a soaked and sticky appearance.

An alternative route of application based on a deposit on the skin (“spot-on” or “pour-on”) is advantageous by virtue of its efficiency and of the rapidity of its action, and also by virtue of the pleasant appearance of the coat of the animals after application and drying. This type of spot-on application consists in applying, to dry skin, while parting the hairs so as to make the skin clearly visible, a composition in drop form on a zone with a surface area of less than 10 cm², in particular between 5 and 10 cm². After deposit/release, the composition or the compound diffuses then dries. However, the application of spot-on or pour-on type has certain drawbacks. In particular, there is a certain lack of precision with regard to the doses administered since the product can easily be lost by running on the hair. It is therefore necessary to perform one or more deposits on the back of the animal in order to be certain of administering an effective dose. The large amount of product deposited in one or more spots on the animal can moreover cause inflammatory-type reactions (erythema or pruritus for example). In addition, the fact of depositing a large amount of product on one or more spots often results in the presence of non-administered product on the hairs of the animal, which can also create risks to the health and safety of the user.

Thus, there remains today, in the animal health field, a need for novel strategies for administering veterinary compounds which make it possible to improve the treatments while at the same time maintaining maximum safety for the user.

The inventors have developed a novel approach for treating animals, which consists in topically and sequentially applying a veterinary composition. The inventors have shown that this sequential topical application has advantages, in particular for treatments of parasites, compared with spot-on or pour-on application, such as the prevention of side effects and efficacy.

### SUMMARY OF THE INVENTION

The inventors have developed a novel method for treating animals which consists in administering a veterinary composition or compound topically and sequentially according to a determined application mode. The inventors have shown that this method allows better control of the doses and greater efficacy of action, without side effects for the animals. In particular, the inventors have shown, surprisingly, that the sequential administration with an initial dose (termed loading dose) of veterinary compounds can make it possible to obtain, at equal total doses, an effect greater than that obtained in particular by spot-on or pour-on application.

A subject of the present invention therefore lies in a process for applying a veterinary compound to a non-human mammal, comprising the sequential topical application of a determined total dose of said compound according to a dosage regimen comprising (i) an initial dose D0, applied at the beginning of treatment, representing at most 65% of the determined total dose, and (ii) a plurality of maintenance doses Di, applied sequentially during the duration of the treatment. The sum of the Di doses and of the D0 dose is equal to the determined total dose.

Another subject of the present invention lies in a process for therapeutic or cosmetic treatment of a non-human mammal by administration of a compound, the process comprising the sequential topical application of a determined total dose of said compound according to a dosage regimen comprising (i) an initial dose D0, applied at the beginning of treatment, representing at most 65% of the determined total dose, and (ii) a plurality of maintenance doses Di, applied sequentially during the duration of the treatment. The sum of the Di doses and of the D0 dose is equal to the determined total dose.

Another subject of the invention relates to a compound for use thereof for the therapeutic or cosmetic treatment of a non-human mammal by sequential topical application of a determined total dose of said compound, characterized in that the application is carried out according to a dosage regimen comprising (i) an initial dose D0, applied at the beginning of treatment, representing at most 65% of the determined total dose, and (ii) a plurality of maintenance doses Di, applied sequentially during the duration of the treatment. The sum of the Di doses and of the D0 dose is equal to the determined total dose.

Another subject of the invention relates to the use of a compound for producing a composition intended for the therapeutic or cosmetic treatment of a non-human mammal by sequential topical application of a determined total dose of said compound, characterized in that the application is carried out according to a dosage regimen comprising (i) an initial dose D0, applied at the beginning of treatment, representing at most 65% of the determined total dose, and (ii) a plurality of maintenance doses Di, applied sequentially during the duration of the treatment. The sum of the Di doses and of the D0 dose is equal to the determined total dose.

The compound (or a composition comprising the compound) can be applied to the skin or the hair of the non-human mammal manually (pipette, spray) or in an automated manner.

Another subject of the invention relates to a device for delivering a compound to a non-human mammal, characterized in that it comprises a determined total dose of said compound and a controlled sequential release system ensuring the delivery of an initial dose D0 of the compound at the beginning of treatment, said dose D0 representing 0.5% to 65% of the determined total dose, and of a plurality of maintenance doses Di of the compound, the sum of the Di doses and of the D0 dose being equal to the determined total dose. The device may be for example a pipette or a collar, in particular a collar comprising a dispersing nozzle, etc.

The invention is applicable to any veterinary compound (or mixture of compounds), and in particular antiparasitic, cosmetic or dermatological compounds. It can be implemented in any non-human mammal, such as in particular pets (canines, felines, etc.), farm animals (cattle, the ovine race, pigs, etc.), horses, etc.

### FIGURE LEGEND

FIG. 1: Compared efficacy of an antiparasitic applied topically by the spot-on technique or by the method according to the invention.

### DETAILED DESCRIPTION OF THE INVENTION

The present invention relates to compositions, methods and apparatuses/devices for treating non-human mammals. It relates more particularly to methods of administration or of treatment based on the topical and sequential application, according to a determined application mode, of a compound for veterinary use, and also to compositions and apparatuses suitable for the implementation thereof. The inventors have shown that this method of administration allows better treatment adherence, without side effects for the animals.

A subject of the invention therefore relates to a compound (or a composition) for use thereof for treating a non-human mammal by sequential topical application of a determined total dose of said compound, characterized in that the application is carried out according to a dosage regimen comprising (i) an initial dose D0, applied at the beginning of treatment, representing at most 65% of the determined total dose, and (ii) a plurality of maintenance doses Di, applied sequentially during the duration of the treatment, the sum of the Di doses and of the D0 dose being equal to the determined total dose.

### Protocol

As indicated, the invention is based in particular on the surprising demonstration that the distribution of the effective total amount of a compound for veterinary use in several sequential doses (or fractions), including an initial dose (loading dose D0) and maintenance doses (Di or di), makes it possible to obtain a better effect than the topical application of the determined total dose (D).

Thus, for a determined total dose, the protocol uses fractions administered sequentially according to the following formula:

$$D = D 0 + {\sum }_{i = 0}^{n} di$$

in which:
D: determined total dose,
D0: initial dose (loading dose),
dᵢ or Di: maintenance doses, and
i the number of sequential applications of the maintenance dose.

For a given compound/composition, the determined total dose typically corresponds to the effective dose recommended by the practitioner, which is generally known in the literature and/or determined by those skilled in the art on the basis of their general knowledge or by carrying out preliminary or clinical tests. For example, for an antiparasitic which acts against fleas and ticks, such as Frontline® Combo (mixture of fipronil and S-methoprene sold by the company Merial), the determined total dose will typically be 5 mg of fipronil and 6 mg of S-methoprene per kg of bodyweight for a cat, and 6.7 mg of fipronil and 6 mg of S-methoprene per kg of bodyweight for a dog. The determined total dose can thus vary according to the non-human mammal under consideration, its weight and of course the nature of the compound for veterinary use. For the treatment according to the invention, the determined total dose is distributed in several fractions that will be administered sequentially, first of all an initial dose termed loading dose (D0), then a plurality of maintenance doses Di.

The initial dose (D0) may be adapted by those skilled in the art according to the class or nature of the compound/composition applied, to the target non-human mammal and to the duration of the treatment.

Nevertheless, in one preferred embodiment, the initial dose D0 represents from 0.5% to 60% of the determined total dose, preferably from 1% to 60%. More preferentially, when the targeted treatment duration is less than 2 months, the initial dose D0 advantageously represents from 10-60% of the determined total dose, in particular from 15-60%, from 20-60%, from 20 to 50% or from 30 to 50%. When the targeted treatment duration is greater than 2 months, the initial dose D0 preferably represents from 0.5% to 10%, from 0.5% to 8%, more preferentially from 1% to 5%, of the determined total dose.

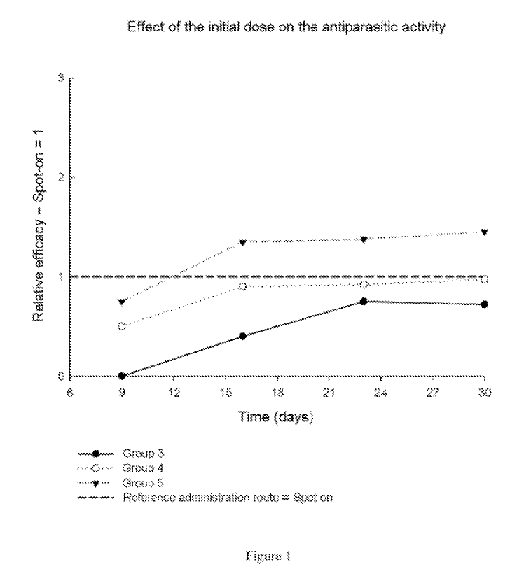

As shown by the results presented in FIG. 1, for an antiparasitic composition such as Vectra3D (Ceva Santé Animale), loading doses of 30% or 50% give an effect greater than the reference topical treatment of spot-on type, whereas, without loading dose, the results remain inferior. In addition, with a loading dose representing 20% of the total dose, the efficacy results observed show a continuous increase in the anti-flea efficacy over time, which is reflected by an increased treatment efficacy compared with a topical application of spot-on type.

In one particular mode, when the compound is a therapeutic agent (for example an antiparasitic or a mixture of antiparasitics) with a targeted treatment duration of less than 2 months, the initial dose D0 advantageously represents from 10-60% of the determined total dose, preferably from 15-60%, more preferentially from 20% to 60%, from 20% to 50% or even more preferentially from 30-50%.

In one particular mode, when the compound is a cosmetic or dermatological agent (for example an essential oil or a mixture of essential oils) with a treatment duration of greater than 2 months, the initial dose D0 advantageously represents from 0.5% to 10% of the determined total dose, from 0.5% to 8%, or more preferentially from 1-5%.

After application or release of the initial dose, the treatment of the invention comprises the application or the release of several maintenance doses (Di). In the context of the invention, the maintenance doses may be identical or variable during treatment. Preferably nevertheless, all the maintenance doses Di are identical.

Preferably, each maintenance dose Di represents at most 35% of the initial dose D0, more preferentially at most 20%, even more preferentially at most 10%. Moreover, the frequency of application of the maintenance doses Di may be constant throughout the treatment, or variable.

In one preferred mode, the maintenance doses Di are identical and their frequency of application is constant during the treatment. Advantageously, the frequency of application of the maintenance doses Di is between 1 h and 1 month, preferably between 2 h and 15 days, more preferentially between 4 h and 7 days, even more preferentially between 12 h and 4 days.

The duration of the treatment, or the frequency of application and/or the amount of the maintenance doses Di can be adjusted by those skilled in the art according to the compound, to the non-human mammal and to the type of treatment. Thus, the duration of the treatment can be between 1 and 24 months, more preferentially between 1 and 18 months, for example between 6 and 12 months or 1 and 6 months.

In one particular mode, the compound is a therapeutic agent (e.g., antiparasitic) or a mixture of therapeutic agents (e.g. of antiparasitics), the initial dose D0 represents from 20-60% of the determined total dose, and each maintenance dose Di is identical and administered daily.

In another particular mode, the compound is a cosmetic or dermatological agent (e.g. an essential oil) or a mixture of cosmetic or dermatological agents (e.g. a mixture of essential oils), the initial dose D0 represents from 1-5% of the determined total dose, and each maintenance dose Di is identical and administered every 3 days.

### Topical Administration

The invention comprises the sequential topical administration of one or more compounds, according to a determined application mode. The administration is topical and preferentially localized on the skin, at the surface.

In one preferred mode of the invention, the compound or the composition is administered on a zone of the animal's skin, for example the neck or any practical zone (for example the back, between the shoulders, etc.) by means of a device suitable for the targeted zone, such as, for example, a collar, a spray or a dispersing nozzle. The administration can be carried out by means of any device comprising a container containing the compound.

In one preferred mode, the compound or the composition is administered by means of a controlled-release device, for example a collar. Other controlled-release-mode devices are mentioned for example in patents U.S. Pat. No. 7,140,325 or U.S. Pat. No. 6,010,492.

The administration of the compound or the composition is generally continued until the whole of the determined total dose is administered. Of course, the treatment can be interrupted if the purpose is achieved beforehand or if the practitioner decides to do so.

### Compound

The term “compound” is intended to mean in particular any therapeutic, cosmetic or dermatological agent that can be administered to a non-human animal topically. It may therefore be, for example, an antiparasitic agent, or a cosmetic or dermatological agent. The compound may be isolated or in the form of a mixture with one or more other compounds.

The term “therapeutic agent” is intended to mean any substance or mixture which makes it possible to block, prevent, eliminate or decrease the number of ectoparasites or of endoparasites on animals, and/or to prevent an infestation of ectoparasites and/or of endoparasites on non-human mammals.

The therapeutic agents according to the invention may be of natural or synthetic origin or a combination of both. These therapeutic agents comprise in particular insect growth regulators, antibiotic pesticides, botanical pesticides, organophosphorus compounds, carbamates, amidines, organochlorinated compounds, phenylpyrazole compounds, pyrethroids, benzylureas, pyrethrinoids, formamidines, semicarbazones, neonicotinoids, diamides, spinosyns, isoxazolines, copper-containing pesticides, anthelmintic agents, benzimidazoles, pro-benzimidazoles, salicylanilides, imidazothiazoles, amino-acetonitrile derivatives (AADs), spiroindoles, pyrimidines, substituted phenols, and anticoccidials.

For example, the insect growth regulator may be a chitin synthesis inhibitor or a synoptic juvenile growth hormone, such as azadirachtin, bistrifluron, diofenolan, fenoxycarb, hydroprene, kinoprene, methoprene, pyriproxyfen, tetrahydroazadirachtin, chlorfluazuron, cyromazine, diflubenzuron, fluazuron, flucycloxuron, flufenoxuron, hexaflumuron, lufenuron, novaluron, noviflumuron, tebufenozide, teflubenzuron, or triflumuron. The antibiotic pesticide may be a Bacillus thuringiensis toxin. The botanical pesticides may be d-limonene, thymol, geraniol, linalol, carvacrol, nicotine, ryania or pyrethrins. The organophosphorus compounds may be chosen from dicrotophos, terbufos, dimethoate, diazinon, disulfoton, trichlorfon, azinphos-methyl, chlorpyrifos, malathion, oxydemeton-methyl, methamidophos, acephate, ethyl-parathion, parathion-methyl, mevinphos, phorate, carbofenthion, phosalone, naphthalophos or pyraclofos. The carbamates may be chosen from carbaryl, carbofuran, aldicarb or carbofuran. The organochlorinated compounds may be chosen from methoxychlor, dicofol or a cyclodiene such as endosulfan. The pyrethrinoids may be chosen from allethrin, resmethrin, permethrin, flumethrin, deltamethrin and cypermethrin. An example of formamides is amitraz. An example of semicarbazones is metaflumizone. The neonicotinoids may be chosen from imidacloprid, nitenpyram or dinotefuran. The copper-containing pesticide may be copper(II), or a copper oxychloride hydroxide or sulfate, namely (Cu₂Cl(OH)₃) mixed with (Cu₄(OH)₆(SO₄)). The anthelmintic agent may be chosen from a macrocyclic lactone such as an avermectin (for example ivermectin and moxidectin) or a milbemycin (for example milbemycin oxime). The benzimidazoles may be albendazole or thiabendazole. The salicylanilides may be closantel or oxyclozanide. Levamisole may be one of the imidazothiazoles. Pyrantel may be one of the pyrimidines. Nitroxynil may be one of the substituted phenols. Fipronil may be one of the phenylpyrazoles. The isoxazolines may be 4-{5-[3-chloro-5-(trifluoromethyl)phenyl]-5-(trifluoromethyl)-4,5-dihydroisoxazol-3-yl}-N-{2-oxo-2-[(2,2,2-trifluoroethyl)amino]ethyl}naphthalene-1-carboxamide (afoxolaner), 4-[5-(3,5-dichlorophenyl)-5-(trifluoromethyl)-4,5-dihydro-1,2-oxazol-3-yl]-2-methyl-N-{2-oxo-2-[(2,2,2-trifluoroethyl)amino]ethyl}benzamide (fluranaler) or N-{2-chloro-{5-(3,5-dichlorophenyl)-5-trifluoromethyl-4,5-dihydroisoxazol-3-yeenzyl}cyclopropanecarboxylic.

In one particular embodiment of the invention, the compound is a therapeutic agent which comprises a neonicotinoid, preferably dinotefuran, a pyrethrinoid, preferably permethrin or flumethrin, and an IGR, preferably pyriproxyfen.

In other particular embodiment of the invention, the compound is a therapeutic agent which comprises a phenylpyrazole, preferably fipronil, and an IGR, preferably methoprene.

The various classes of parasites and classes of antiparasitic agents and also the active agents sold can be found on the parasitipedia website at the following address: http://parasitipedia.net/; and on the European Scientific Counsel Companion Animal Parasite (ESCCA) website at the following address: http://www.esccap.org/.

The term “cosmetic or dermatological agent” is intended to mean a substance or a mixture for the purpose, exclusively or mainly, of cleaning, fragrancing, modifying the appearance, protecting, treating, preventing or maintaining in good health the superficial parts (epidermis, hair systems, etc) or correcting the body odors of non-human mammals. The cosmetic or dermatological agents according to the invention may be of natural or synthetic origin or a combination of both.

By way of example, mention may be made of moisturizing agents, emollients, fragrances, plant extracts, algal extracts, vitamins, essential oils or antibiotics.

### Composition

The term “composition” is intended to mean any formulation used for applying a compound, whether it is pure or in mixture form, additionally comprising a carrier or excipient that is acceptable from a veterinary point of view.

The term “excipient that is acceptable from a veterinary point of view” is intended to mean an excipient that is tolerated by the non-human mammal when it is applied topically, and which is capable of sufficiently dissolving and/or formulating the compound.

The composition is preferentially a liquid form preferably comprising one or more organic solvents such as, in particular, a C1-C4 alkyl alcohol, such as ethanol, isopropanol or methanol; acetyl tributyl citrate; one or more fatty acid esters such as dimethyl ester and diisobutyl adipate; acetone, acetonitrile, benzyl alcohol, butyldiglycol, dimethylacetamide, dimethylformamide, dipropylene glycol n-butyl ether, ethylene glycol monoethyl ether, ethylene glycol monomethyl ether, diethylene glycol monoethyl ether, monomethylacetamide, dipropylene glycol monomethyl ether, liquid polyoxyethylene glycols, propylene glycol, 2-pyrrolidones such as N-methyl-pyrrolidone and N-octyl-2-pyrrolidone, propylene carbonate, diethylene glycol monoethyl ether, dimethyl sulfoxide, ethylene glycol, diethyl phthalate, ethoxydiglycol, or combinations thereof.

In one embodiment, the organic solvent comprises N-methylpyrrolidone and N-octyl-2-pyrrolidone. In one preferential embodiment, the organic solvent comprises isopropanol, isopropyl myristate and medium-chain triglycerides, alone or combined. In one even more preferential embodiment, the organic solvent comprises dimethyl sulfoxide.

The composition of the present invention may also comprise one or more additional agents or adjuvants, such as one or more co-solvents, dyes, spreading agents, antioxidants, light-stabilizers and/or adhesion agents.

### Non-Human Mammals

The invention is suitable for the treatment of any non-human mammal. Preferably, the non-human mammal belongs to the pets group (preferentially a dog or a cat) or is chosen from farm animals (cattle, the ovine race, the equine race, pigs, etc.), such as cows, horses, pigs, etc.

Other aspects and advantages of the invention will emerge on reading the examples which follow, which should be considered to be nonlimiting illustrations.

### EXAMPLES

### Example 1

### Study of Efficacy of a One-Month Treatment With an Antiparasitic on Ticks

Forty (40) dogs weighing 10 to 25 kg were included in the study and separated into five groups:
Group 1: Control group consisting of 8 untreated dogs.
Group 2: “Spot-on” group or “reference administration group: spot-on” group consisting of 8 dogs. 3.6 ml (=D: Determined total dose) of Vectra 3D (Ceva Santé Animale) were administered to each dog by “spot on” in three spots (between the shoulder blades, in the middle of the back and at the base of the tail) on day Dzero.
Group 3: This group consists of 8 dogs. A total volume of 3.6 ml (100% of D) of Vectra 3D is continuously administered to each dog from Dzero to D28 at a rate of approximately 0.124 ml per day.
Group 4: This group consists of 8 dogs. Each dog is administered an initial dose of 0.72 ml (20% of D) of Vectra 3D on Dzero, followed by 2.88 ml continuously in identical daily doses from D1 to D28 (0.10 ml per day).
Group 5: This group consists of 8 dogs. Each dog is administered an initial dose of 1.08 ml (30% of D) of Vectra 3D on Dzero, followed by 2.52 ml continuously in identical daily doses from D1 to D28 (0.09 ml per day).

All 40 dogs of this study are infested with 50 (±4) adult Dermacentor reticulatus (D. reticulatus) ticks on D-7 and then on D-2, D7, D14, D21 and D28. The ticks are counted and removed 2 days (±2 h) after the infestation, that is to say on D-5, Dzero, D9, D16, D23 and D30. For each dog, the number of dead ticks at each spot (D-5, Dzero, D9, D16, D23 and D30) is determined. It is equal to the number of ticks implanted (50) minus the number of live ticks (gorged with blood) counted and removed at the observation points. An average number of dead ticks is then calculated by calculating the arithmetic mean for each group.

The relative efficacy is then calculated as the ratio of the number of ticks killed in the treated groups 2, 3, 4 and 5 relative to the number of ticks counted in the control group. The efficacy results for groups 2, 3, 4 and 5 are presented in FIG. 1.

During this study, none of the dogs presented in the treated groups (groups 2, 3, 4 and 5) exhibited any sign of inflammation at the application points. Furthermore, no clinical sign linked to the treatments was observed during this trial.

Results:

With an initial dose D0 of 30% followed by identical daily maintenance doses, a significant difference is obtained compared with a “spot on” application. It can be concluded that the antiparasitic effect produced by the administration mode according to the present invention is stronger than that obtained with a “spot-on” application. These results illustrate the efficacy and unexpected nature of the method of the invention which, at equal total dose, demonstrates a greater antiparasitic effect.

### Example 2

### Study of Efficacy for a Treatment Duration Greater than 2 Months with a Dermo-Cosmetic Preparation

A mixture of essential oils (0.5% rosemary and 0.5% lavender) dispersed in isopropyl myristate is prepared. A determined total dose of 31 ml of this mixture is applied to a group of 8 dogs according to the following protocol:

Application of an initial dose of 1 ml of a mixture of essential oils at the base of the neck, then a maintenance dose of 0.25 ml (0.8%) at the base of the neck every three days for 360 days.

The control group is composed of 8 dogs having received no treatment.

The appearance of the coat (sheen and silky feel) of the treated animals is substantially improved after the treatment.

1. A method for treating a non-human mammal, comprising administering to the skin a determined total dose of a compound, wherein the administration is carried out according to a dosage regimen comprising (i) an initial dose D0, administered at the beginning of treatment, representing at most 65% of the determined total dose, and (ii) a plurality of maintenance doses Di, administered sequentially during the duration of the treatment, the sum of the Di doses and of the D0 dose being equal to the determined total dose.

2. The method as claimed in claim 1, wherein the initial dose D0 represents 0.5% to 60% of the determined total dose.

3. The method as claimed in claim 1, wherein all the maintenance doses Di are identical.

4. The method as claimed in claim 1, wherein each maintenance dose Di represents at most 35% of the initial dose D0.

5. The method as claimed in claim 1, wherein the frequency of administration of the maintenance doses Di is constant throughout the treatment.

6. The method as claimed in claim 1, wherein the frequency of administration of the maintenance doses Di is between 1 h and 1 month.

7. The method as claimed in claim 1, wherein the duration of the treatment is between 1 month and 24 months.

8. The method as claimed in claim 1, wherein the compound is formulated in a composition with at least one carrier or excipient that is acceptable.

9. The method as claimed in claim 1, wherein the compound is administered by means of a controlled-release device.

10. The method as claimed in claim 9, wherein the compound is applied by means of a collar.

11. The method as claimed in claim 1, wherein the compound is a therapeutic agent, a mixture of therapeutic agents, a therapeutic cosmetic agent, a cosmetic or dermatological agent, or a mixture of cosmetic or dermatological agents.

12. The method as claimed in claim 1, wherein the initial dose D0 represents from 10-60% of the determined total dose, wherein each maintenance dose Di is identical and administered daily and wherein the duration of the treatment is from 1 to 2 months.

13. The method as claimed in claim 11, wherein the compound is a therapeutic agent or a mixture of therapeutic agents.

14. The method as claimed in claim 13, wherein the therapeutic agent is selected from the group consisting of an insect growth regulator, an antibiotic pesticide, a botanical pesticide, an organophosphorus compound, a carbamate, an amidine, an organochlorinated compound, a phenylpyrazole, a pyrethroid, a benzylurea, a pyrethrinoid, a formamidine, a semicarbazone, a neonicotinoid, a diamide, a benzimidazole, pro-benzimidazoles, a spinosyn, an isoxazoline, a copper-containing pesticide, an anthelmintic agent, a salicylanilide, a substituted phenol, a pyrimidine, an amino-acetonitrile derivative, a spiroindole, an imidazothiazole, an anticoccidial, and a mixture thereof.

15. The method as claimed in claim 1, wherein the initial dose D0 represents from 0.5-10% of the determined total dose, wherein each maintenance dose Di is identical and administered every 3 days and wherein the preferential duration of the treatment is greater than 2 months.

16. The method as claimed in claim 11, wherein the compound is a cosmetic or dermatological agent or a mixture of cosmetic or dermatological agents.

17. A device for delivering a compound to a non-human mammal, wherein the device comprises a determined total dose of said compound and a controlled sequential release system ensuring the delivery of an initial dose D0 of the compound at the beginning of the treatment, said dose D0 representing 0.5% to 65% of the determined total dose, and of a plurality of maintenance doses Di of the compound, the sum of the Di doses and of the D0 dose being equal to the determined total dose.

18. A method for applying a cosmetic compound to a non-human mammal, comprising the topical administration of a determined total dose of said compound according to a dosage regimen comprising (i) an initial dose D0 representing 0.5% to 65% of the determined total dose, and (ii) a plurality of maintenance doses Di, applied sequentially after the initial dose, the sum of the Di doses and of the D0 dose being equal to the determined total dose.

19. The method as claimed in claim 1, wherein each maintenance dose Di represents at most 20% of the initial dose D0.

20. The method as claimed in claim 1, wherein each maintenance dose Di represents at most 10% of the initial dose D0.

21. The method as claimed in claim 1, wherein the frequency of administration of the maintenance doses Di is between 2 h and 15 days.

22. The method as claimed in claim 1, wherein the frequency of administration of the maintenance doses Di is between 4 h and 7 days.

23. The method as claimed in claim 1, wherein the frequency of administration of the maintenance doses Di is between 12 h and 96 h.

24. The method as claimed in claim 1, wherein the duration of the treatment is between 1 and 2 months.

25. The method as claimed in claim 1, wherein the duration of the treatment is between 2 and 12 months.
